(19) United States
(12) Patent Application Publication
(10) Pub. No.: US 2023/0301549 A1
(43) Pub. Date: Sep. 28, 2023

(54) OPERATION APPARATUS AND OPERATION ESTIMATION METHOD

(71) Applicant: Murata Manufacturing Co., Ltd., Nagaokakyo-shi (JP)

(72) Inventors: Toshio TSUJI, Higashi-Hiroshima City (JP); Akira FURUI, Higashi-Hiroshima City (JP); Shumma JOMYO, Higashi-Hiroshima City (JP); Tomomi TSUNODA, Nagaokakyo-shi (JP); Tatsuhiko MATSUMOTO, Nagaokakyo-shi (JP)

(21) Appl. No.: 18/327,285

(22) Filed: Jun. 1, 2023

Related U.S. Application Data
(63) Continuation of application No. PCT/JP2021/029466, filed on Aug. 10, 2021.

(30) Foreign Application Priority Data
Dec. 14, 2020 (JP) 2020-206397

(51) Int. Cl. A61B 5/11; A61B 5/00

(52) U.S. Cl. CPC A61B 5/1118; A61B 5/6824; A61B 5/746; A61B 5/742

(57) ABSTRACT

An operation apparatus is provided that includes a plurality of sensors, a range setting portion, and an arithmetic portion. The plurality of sensors are worn on a wrist, and output a sensor signal according to a motion of a tendon of the wrist. The range setting portion sets an operation learning time range including time of a feature point of a measurement signal based on the sensor signal. The arithmetic portion learns an operation based on the measurement signal based on the sensor signal of the plurality of sensors in the operation learning time range. The arithmetic portion then estimates the operation based on criteria according to the learned content.

### CROSS REFERENCE TO RELATED APPLICATIONS

This application is a continuation of PCT Application No. PCT/JP2021/029466, filed Aug. 10, 2021, which claims priority to Japanese Patent Application No. 2020-206397, filed Dec. 14, 2020, the entire contents of each of which are hereby incorporated by reference in their entirety.

### TECHNICAL FIELD

The present invention relates to a technology for detecting an operation from an action of a hand.

### BACKGROUND

Japanese Unexamined Patent Application Publication No. 2005-352739 (hereinafter “Patent Literature 1”) discloses a portable terminal device that uses a piezoelectric sensor. The portable terminal device disclosed in Patent Literature 1 places a plurality of piezoelectric sensors on the back side of a wrist.

The portable terminal device disclosed in Patent Literature 1 measures a motion of a finger of a user (e.g., a wearer), by using a detection signal of a plurality of piezoelectric elements.

However, in the conventional configuration such as the portable terminal device disclosed in Patent Literature 1, it is difficult to measure the motion of a finger with high accuracy.

### SUMMARY OF THE INVENTION

In view of the foregoing, an operation estimation technology is provided for measuring a motion (e.g., an operation with a finger) of a finger with high accuracy.

In an exemplary aspect, an operation apparatus is provided that includes a plurality of sensors, a range setting portion, and an arithmetic portion. The plurality of sensors are worn on a wrist, and output a sensor signal according to displacement of a body surface of the wrist. The range setting portion sets an operation learning time range including time of a feature point of the sensor signal of the plurality of sensors. Moreover, the arithmetic portion estimates an operation based on the sensor signal of the plurality of sensors in the operation learning time range.

In this configuration, by use of a characteristic part of the sensor signal according to the displacement (e.g., a motion of the tendon of the wrist) of the body surface of the wrist, an operation is learned with high accuracy, and the operation is estimated using this learning result. In addition, the displacement (e.g., the motion of the tendon of the wrist) of the body surface of the wrist is closely linked to a motion of a finger. Consequently, estimation accuracy for the operation with a finger is increased.

According to the present invention, an operation with a finger can be detected with high accuracy.

### BRIEF DESCRIPTION OF DRAWINGS

FIG. 1 is a functional block diagram showing an example of a configuration of an operation apparatus according to a first exemplary embodiment.

FIG. 2A and FIG. 2B are views showing a specific configuration and a wearing example of a strain sensor.

FIG. 3 is a graph showing an example of a waveform of a measurement signal.

FIG. 4 is a functional block diagram showing an example of a configuration of an estimation portion according to the first exemplary embodiment.

FIG. 5 is a functional block diagram showing an example of a configuration of an index value calculation portion.

FIG. 6A and FIG. 6B are charts showing a concept of a total activity level.

FIG. 7 is a functional block diagram showing an example of a configuration of a range setting portion according to the first exemplary embodiment.

FIG. 8 is a waveform diagram of the total activity level used for range setting.

FIG. 9 is a functional block diagram showing an example of a configuration of an arithmetic portion according to the first exemplary embodiment.

FIG. 10 is a flow chart showing an example of an operation estimation method according to the first exemplary embodiment.

FIG. 11 is a graph illustrating a concept of estimation.

FIG. 12 shows a concept in a case in which a combined operation is determined.

FIG. 13 shows a concept in the case in which a combined operation is determined.

FIG. 14 shows a concept in the case in which a combined operation is determined.

FIG. 15 is a flow chart showing an example of the operation estimation method according to the first exemplary embodiment.

FIG. 16 is a view showing an example of an application target of the operation apparatus according to the present exemplary embodiment.

FIG. 17 is a functional block diagram showing an example of a configuration of an operation apparatus according to a second exemplary embodiment.

FIG. 18 is a functional block diagram showing an example of a configuration of an operation apparatus according to a third exemplary embodiment.

FIG. 19 is a view showing a wearing example of the operation apparatus according to the third exemplary embodiment.

FIG. 20 is a functional block diagram showing an example of a configuration of an operation apparatus according to a fourth exemplary embodiment.

FIG. 21 is a functional block diagram showing an example of a configuration of an operation apparatus according to a fifth exemplary embodiment.

FIG. 22 is a functional block diagram showing an example of a configuration of an arithmetic portion that only estimates an operation.

### DETAILED DESCRIPTION OF EXEMPLARY EMBODIMENTS

### First Exemplary Embodiment

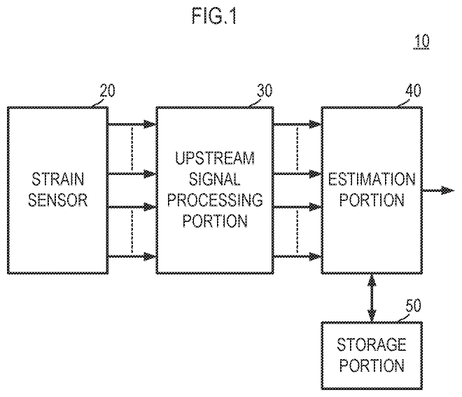

An operation estimation technology according to a first exemplary embodiment will be described with reference to the drawings. FIG. 1 is a functional block diagram showing an example of a configuration of an operation apparatus according to the first exemplary embodiment.

As shown in FIG. 1, the operation apparatus 10 includes a strain sensor 20, an upstream signal processing portion 30, an estimation portion 40, and a storage portion 50. In an exemplary aspect, the upstream signal processing portion 30, the estimation portion 40, and the storage portion 50 are formed by an electronic component, an electronic circuit, or the like, and are built in a predetermined housing, for example.

(Configuration and Processing of Strain Sensor 20)

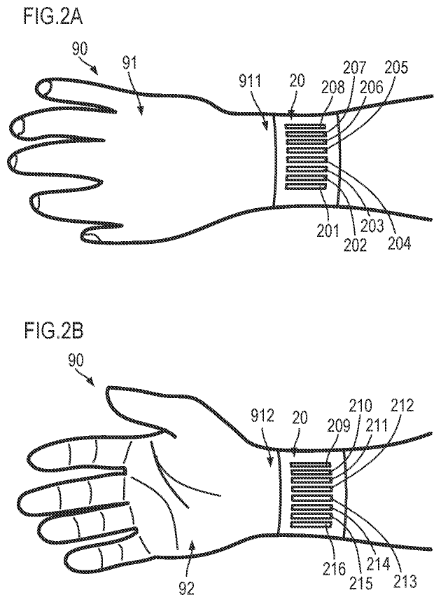

FIG. 2A and FIG. 2B are views showing a specific configuration and a wearing example of the strain sensor. FIG. 2A shows the front side of a hand and a wrist, and FIG. 2B shows the back side of the hand and the wrist.

As shown in FIG. 2A and FIG. 2B, the strain sensor 20 is worn on a wrist and includes a plurality of sensors 201 to 216. The plurality of sensors 201 to 216 include a configuration in which a detection electrode is disposed on a piezoelectric film with flexibility. The piezoelectric film is, for example, made of polylactic acid as a main component and is extended in a predetermined direction.

As more specific placement, the plurality of sensors 201 to 208 are worn on a front surface 911 of a wrist. The front surface 911 of a wrist is a surface near a back 91 of a hand in the wrist. The plurality of sensors 201 to 208 are spaced apart in a circumferential direction of the wrist and are worn on the front surface 911 of the wrist so that a longitudinal direction of the piezoelectric film and the electrode may be parallel to a direction in which the tendon of the wrist is extended.

The plurality of sensors 209 to 216 are worn on a back surface 912 of the wrist. The back surface 912 of the wrist is a surface near a palm 92 of a hand in the wrist. The plurality of sensors 209 to 216 are spaced apart in the circumferential direction of the wrist. The plurality of sensors 209 to 216 are worn on the back surface 912 of the wrist so that the longitudinal direction of the piezoelectric film and the electrode may be parallel to a direction in which the tendon of the wrist is extended. The strain sensor 20 may include lead-out wiring for outputting an obtained sensor signal to outside, which is omitted from illustration in FIG. 2A and FIG. 2B. It is noted that while the plurality of sensors are shown to be sixteen total sensors, there can be more or less sensors in alternative aspects.

It is also noted that, when the piezoelectric film of the plurality of sensors 201 to 216 is made of PLLA of polylactic acid, a direction of extension may be approximately 45 degrees to a direction in which the tendon of a wrist is extended. It is also noted that, in the present exemplary embodiment, a shape of the electrode is not only a rectangle, but also may be another shape such as a square or a circle. In addition, the material of the piezoelectric film is not limited to polylactic acid. Moreover, mainly due to the ability to follow the body surface, a film-shaped piezoelectric element is preferred but not required.

When a wearer of the strain sensor 20 moves a finger, according to a motion of the finger, the tendon of the wrist moves and the body surface is displaced. For example, in a case in which the wearer operates a virtual keyboard to be described below, according to the motion of fingers, the tendon of the wrist moves and the body surface is displaced. Each of the plurality of sensors 201 to 216 of the strain sensor 20, according to the movement (more specifically, the displacement (the displacement of the body surface) of a surface of a skin by the movement of the tendon) of the tendon of the wrist, generates and outputs a sensor signal. The sensor signal is generated with an amplitude according to the magnitude of the movement of the tendon of the wrist, and with a waveform according to time of the movement of the tendon of the wrist. The strain sensor 20 outputs the sensor signal (e.g., the sensor signal of a plurality of detection channels) of the plurality of sensors 201 to 216 to the upstream signal processing portion 30.

According to such a configuration, the strain sensor 20 is configured to output the sensor signal of the plurality of sensors 201 to 216 that is detected with high accuracy, according to the motion of a finger. Furthermore, with this configuration, the strain sensor 20 has flexibility, so that the discomfort of the wearer is able to be reduced, and a reduction in the operability by the wearer is able to be significantly reduced.

(Configuration and Processing of Upstream Signal Processing Portion 30)

The upstream signal processing portion 30 executes direct current component removal processing, amplification processing, A/D conversion processing, and filter processing, on the sensor signal of the plurality of sensors 201 to 216. More specifically, the upstream signal processing portion 30 performs the direct current component removal processing on the sensor signal of the plurality of sensors 201 to 216. The upstream signal processing portion 30 performs the amplification processing on the sensor signal of the plurality of sensors 201 to 216, after the direct current component removal processing. The upstream signal processing portion 30 performs the A/D conversion (analog to digital conversion) processing on the sensor signal of the plurality of sensors 201 to 216, after the amplification processing. It is noted that the order of each processing to be executed by the upstream signal processing portion 30 is not limited to this and can be appropriately set.

The upstream signal processing portion 30 performs the filter processing on a digitalized sensor signal of the plurality of sensors 201 to 216. The filter processing is Nth digital Butterworth low-pass filter processing, for example. The upstream signal processing portion 30 performs normalization processing on the signal on which the filter processing has been performed. It is noted that the normalization processing herein is, for example, processing to unify the reference potential of the sensor signal of the plurality of sensors 201 to 206. The upstream signal processing portion 30 outputs this signal on which the normalization processing has been performed, to the estimation portion 40, as a measurement signal yCH(t) corresponding to the sensor signal of the plurality of sensors 201 to 216. Moreover, the normalization processing, although being able to be omitted, is able to significantly reduce the variation in the measurement signal yCH(t), when used.

Then, through the processing of the above upstream signal processing portion 30, the measurement signal is configured by low-frequency components excluding a direct-current component. Therefore, noise included in the sensor signal can be effectively reduced, and the measurement signal reflects the movement of the tendon with high accuracy.

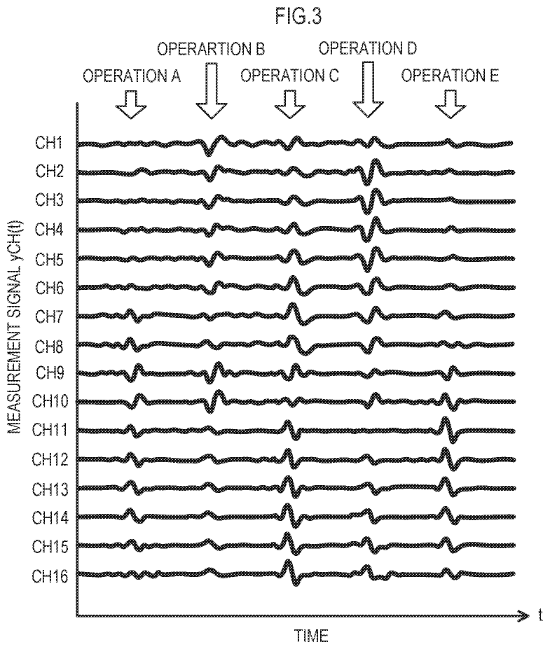

FIG. 3 is a graph showing an example of a waveform of the measurement signal. In FIG. 3, a vertical axis indicates the amplitude of the measurement signal yCH(t) for each channel, and a horizontal axis indicates measurement time. Channels CH1 to CH16 indicated on the vertical axis, that is, measurement signals yCH1(t) to yCH16(t), respectively correspond to sensor signals of the plurality of sensors 201 to 216. In addition, an operation A, an operation B, an operation C, an operation D, and an operation E that are indicated in FIG. 3 each show a case in which a different finger action is performed.

As shown in FIG. 3, due to the difference between the operation A, the operation B, the operation C, the operation D, and the operation E, in other words, when the operations are different, combinations of the waveforms of the measurement signals yCH1(t) to yCH16(t) are different. Therefore, the use of the measurement signals yCH1(t) to yCH16(t) makes it possible to estimate an operation.

(Configuration and Processing of Estimation Portion 40)

The estimation portion 40, roughly, detects the feature point of the measurement signal (the sensor signal) of the plurality of sensors 201 to 216, and estimates an operation, by using the measurement signal (the sensor signal) in an operation estimating time range including time of the feature point. At this time, the estimation portion 40 is configured to estimate an operation, by using estimating database stored in the storage portion 50.

In addition, the estimation portion 40 is configured to perform learning to estimate the operation, by using the measurement signal (the sensor signal) of the plurality of sensors 201 to 216.

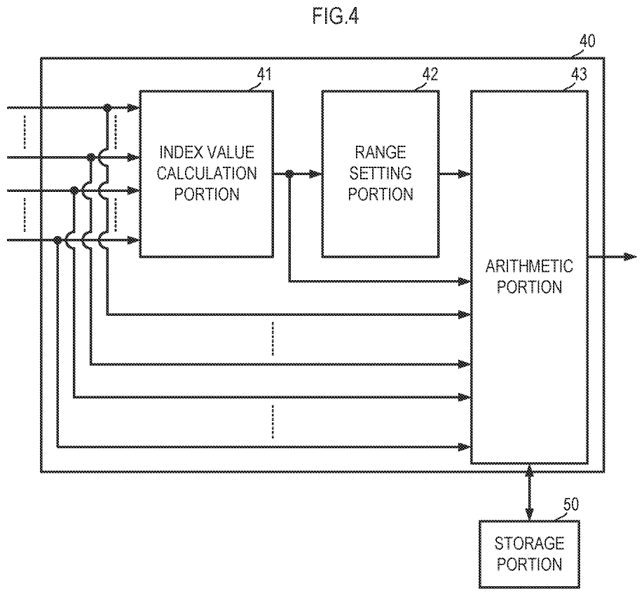

FIG. 4 is a functional block diagram showing an example of a configuration of the estimation portion according to the first exemplary embodiment. As shown in FIG. 4, the estimation portion 40 includes an index value calculation portion 41 (i.e., an index value calculator, a range setting portion 42, and an arithmetic portion 43. In an exemplary aspect, the estimation portion 40 can include a memory and processor (e.g., CPU or microprocessor) configured to execute instructions on the memory so as to perform he algorithms described herein.

In an exemplary aspect, the index value calculation portion 41 calculates a total activity level S(t) being a range setting index, by using the measurement signals yCH1(t) to yCH16(t) of the plurality of sensors 201 to 216.

The range setting portion 42 sets a learning time range, by using a feature point of the total activity level S(t).

The arithmetic portion 43, at time of estimating an operation, estimates the operation, by using operation estimating database stored in the storage portion 50 and the measurement signals yCH1(t) to yCH16(t) in a time window for operation estimation. In addition, the arithmetic portion 43, at time of learning an operation, performs learning for operation estimation, by using the measurement signals yCH1(t) to yCH16(t) in the learning time range.

More specifically, each part of the estimation portion 40 executes the following processing.

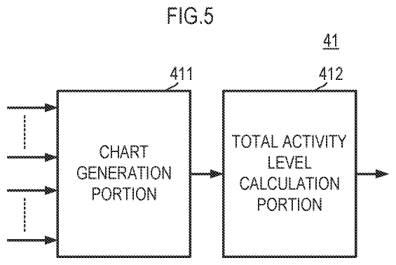

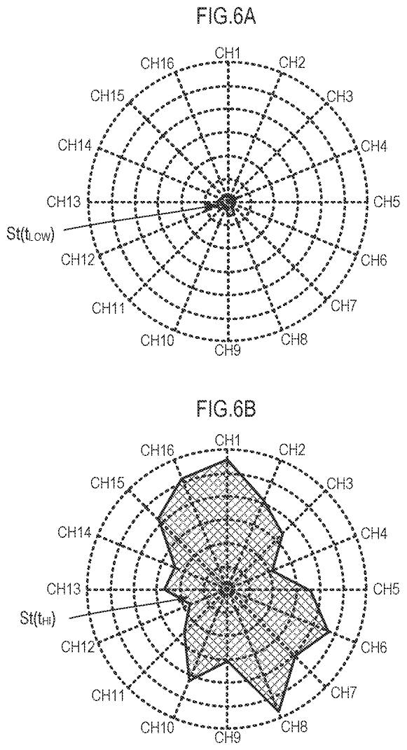

FIG. 5 is a functional block diagram showing an example of a configuration of the index value calculation portion 41. FIG. 6A and FIG. 6B are charts showing a concept of the total activity level. FIG. 6A shows a state (e.g., a Low state) in which an operation is not being performed, and FIG. 6B shows a state (e.g., a Hi state) in which an operation is being performed.

As shown in FIG. 5, the index value calculation portion 41 includes a chart generation portion 411 and a total activity level calculation portion 412 (i.e., a total activity level calculator). The chart generation portion 411 generates a chart diagram, by using the measurement signals yCH1(t) to yCH16(t) of the plurality of sensors 201 to 216. The chart diagram is a diagram in which the plurality of channels CH1 to CH16 corresponding to the measurement signals yCH1(t) to yCH16(t) are placed on the circumference of a circle so that the amplitude may be increased as a distance from a center is increased, the center being set as an absolute value of the amplitude that is set to 0 (zero), and the amplitude (the absolute value) of the measurement signals yCH1(t) to yCH16(t) is plotted for each of the channels CH1 to CH16. In other words, the distance from the center means the magnitude of the measurement signals yCH1(t) to yCH16(t) in each channel.

The chart generation portion 411 generates a chart for the measurement signals yCH1(t) to yCH16(t) at predetermined time intervals (sampling intervals). The chart generation portion 411 outputs a generated chart at each time, to the total activity level calculation portion 412.

The total activity level calculation portion 412 calculates an internal area of the chart as the total activity level S(t). The internal area of the chart is an area of a region (e.g., a region near the center) inside a region to be provided by circumferentially and sequentially connecting plot positions (positions showing the amplitude of the measurement signals yCH1(t) to yCH16(t)) of each channel CH1 to CH16 in the chart.

As shown in FIG. 6A, when an operation is not performed, the amplitude of the measurement signals yCH1(t) to yCH16(t) is small, so that the total activity level S(t) being the internal area of the chart is reduced. In contrast, as shown in FIG. 6B, when an operation is performed, the amplitude of the measurement signals yCH1(t) to yCH16(t) is large, so that the total activity level S(t) being the internal area of the chart is increased. Therefore, the presence or absence of an operation is detectable by use of the magnitude of the total activity level S(t).

The total activity level calculation portion 412 is configured to calculate the total activity level S(t) for each time interval (e.g., a sampling interval for creation of the chart described above) at which the chart generation portion 411 generates the chart, for example. The total activity level calculation portion 412 outputs a calculated total activity level S(t) to the range setting portion 42.

(Configuration and Processing of Range Setting Portion 42)

The range setting portion 42 is mainly used at the time of learning.

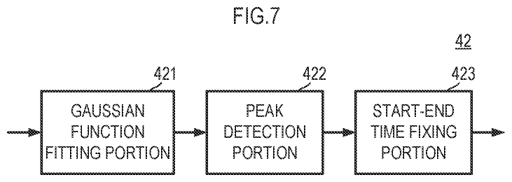

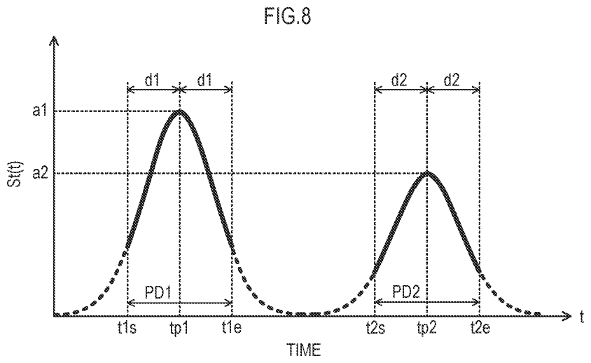

FIG. 7 is a functional block diagram showing an example of a configuration of the range setting portion according to the first exemplary embodiment. FIG. 8 is a waveform diagram obtained by applying Gaussian function fitting to the total activity level used for range setting.

As shown in FIG. 7, the range setting portion 42 includes a Gaussian function fitting portion 421, a peak detection portion 422, and a start-end time fixing portion 423.

The Gaussian function fitting portion 421 fits the total activity level S(t) being a time function, with a Gaussian function showing a normal distribution. As a result, the noise included in the total activity level S(t) is significantly reduced, the total activity level S(t) has a waveform as shown in FIG. 8, and the peaks of the waveform become clearer.

In addition, only any section centered on a peak of the waveform is able to be extracted and used for identification. In the arithmetic portion 43 to be described below, a signal obtained by the action of a finger or a hand and a learning result are used to determine an identification action. Then, in order to identify each action with high accuracy, an appropriate section in which a measurement signal yCH(t) is extracted has to be determined. Therefore, by use of a time waveform (e.g., a time function) of the total activity level S(t) fitted with the Gaussian function, the appropriate section is able to be determined and the identification action to be described below is able to be determined with high accuracy.

The Gaussian function fitting portion 421 outputs the total activity level S(t) after Gaussian function fitting, to the peak detection portion 422.

The peak detection portion 422 detects the peak (the maximum point) and the time of the total activity level S(t) after the Gaussian function fitting. For example, in the example of FIG. 8, the peak detection portion 422 detects a peak value a1 and a peak value a2. The peak value a1 and the peak value a2 each correspond to the “feature point” of the present disclosure.

In addition, the peak detection portion 422 detects a peak time tp1 of the peak value a1, and a peak time tp2 of the peak value a2. The peak detection portion 422 outputs the peak time tp1 and peak time tp2 of the total activity level S(t), to the start-end time fixing portion 423.

The start-end time fixing portion 423 uses the peak time tp1 and the peak time tp2 and fixes a start time and an end time that determine the operation estimating time range.

More specifically, the start-end time fixing portion 423 sets a range setting time d1 with respect to the peak time tp1. The range setting time d1 is set based on spread (e.g., a distribution or the like) of the waveform of the total activity level S (t) at a position at which the peak value a1 is generated, for example. The start-end time fixing portion 423, by subtracting the range setting time d1 from the peak time tp1, sets a learning range start time t1s with respect to the peak value a1. The start-end time fixing portion 423, by adding the range setting time d1 to the peak time tp1, sets a learning range end time t1e with respect to the peak value a1. Then, the start-end time fixing portion 423 sets time from the learning range start time t1s to the learning range end time t1e, as a learning estimating time range PD1.

Similarly, the start-end time fixing portion 423 sets a range setting time d2 with respect to the peak time tp2. The range setting time d2 is set based on the spread (e.g., the distribution or the like) of the waveform of the total activity level S (t) at a position at which the peak value a2 is generated, for example. The start-end time fixing portion 423, by subtracting the range setting time d2 from the peak time tp2, sets a learning range start time t2s with respect to the peak value a2. The start-end time fixing portion 423, by adding the range setting time d2 to the peak time tp2, sets a learning range end time t2e with respect to the peak value a2. Then, the start-end time fixing portion 423 sets time from the learning range start time t2s to the learning range end time t2e, as a learning estimating time range PD2.

It is noted that, in a case in which a plurality of feature points by a plurality of actions are used for identification, fitting is performed with a function configured by the sum of a plurality of Gaussian functions, which thus determines a range in which the measurement signal yCH(t) is extracted. As an example, in a case in which two feature points by two actions shown in FIG. 8 are used to identify one action, fitting is performed with a function configured by the sum of the Gaussian functions of two waveforms, which thus determines the range setting times d1 and d2.

The start-end time fixing portion 423 outputs the learning estimating time range PD1 and the learning estimating time range PD2, to the arithmetic portion 43.

(Configuration and Processing of Arithmetic Portion 43)

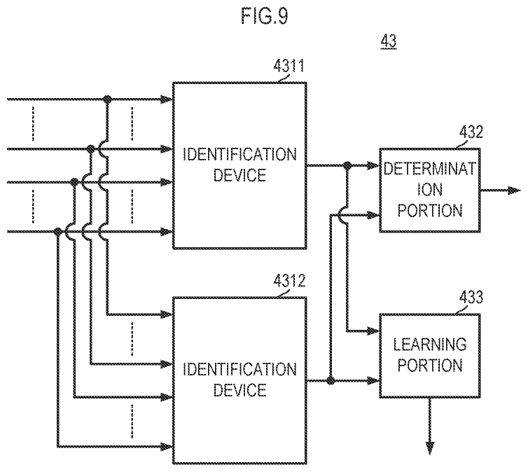

FIG. 9 is a functional block diagram showing an example of a configuration of the arithmetic portion according to the first exemplary embodiment. As shown in FIG. 9, the arithmetic portion 43 includes a plurality of identification devices 4311 and 4312, a determination portion 432, and a learning portion 433.

(During Learning)

The identification device 4311 and the identification device 4312 receive an input of the measurement signals yCH1(t) to yCH16(t) of the plurality of sensors 201 to 216, and a learning time range PD1 and a learning time range PD2. The identification device 4311 and the identification device 4312 obtain a normative signal for identifying an operation, by using the measurement signals yCH1(t) to yCH16(t) in the learning time range PD1 and the learning time range PD2.

The identification device 4311 and the identification device 4312 obtain the normative signal on different conditions. In other words, the identification device 4311 and the identification device 4312 obtain the normative signal to be used for operation estimation in different categories.

For example, the identification device 4311 obtains a normative signal for identifying five fingers individually. The identification device 4312 obtains a normative signal for identifying raising and lowering of a finger.

The identification device 4311 and the identification device 4312 output an obtained normative signal to the learning portion 433.

Moreover, the learning portion 433 stores the obtained normative signal associated with a type of five fingers corresponding to the normative signal and the action of a finger, in the storage portion 50.

As a result, the arithmetic portion 43 can be configured to learn the normative signal according to the type of five fingers and the action of the finger. In a case of such learning, as described above, the measurement signals yCH1(t) to yCH16(t) in the learning time range PD1 and the learning time range PD2 are used to learn by used of the measurement signals yCH1(t) to yCH16(t) that are suitable for learning. As a result, learning accuracy is improved.

In addition, the learning portion 433 can be configured to achieve adaptation of a threshold value Th(t) for action detection during estimation, based on a learned normative signal or the like. As a result, an action is able to be detected with higher accuracy during estimation, which is eventually able to improve estimation accuracy.

(Operation Learning Method)

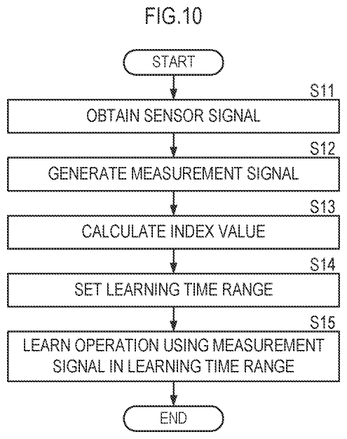

FIG. 10 is a flow chart showing an example of an operation learning method according to the first exemplary embodiment.

The operation apparatus 10 generates a sensor signal according to the movement (e.g., the displacement of the surface of a skin) of the tendon of a wrist by the operation with a finger, by the plurality of sensors 201 to 216 (S11). The operation apparatus 10, by using sensor signals of the plurality of sensors, generates the measurement signals yCH1(t) to yCH16(t), respectively (S12).

The operation apparatus 10, by using the measurement signals of the plurality of sensors, calculates the total activity level S(t) being a range setting index (an index value) (S13). The operation apparatus 10, from time characteristics of the range setting index, detects the feature point of the range setting index, and sets the learning time range (S14). The operation apparatus 10, by using the measurement signals yCH1(t) to yCH16(t) in the learning time range, learns the operation (S15).

(During Estimation)

(1) In a case of setting the operation estimating time range by the Gaussian function fitting and performing operation estimation (e.g., identification and determination)

The Gaussian function fitting portion 421 fits the total activity level S(t) being a time function, with a Gaussian function showing a normal distribution. As a result, the noise included in the total activity level S(t) is significantly reduced, the total activity level S(t) has a waveform as shown in FIG. 8, and the peaks of the waveform become clear.

The Gaussian function fitting portion 421 outputs the total activity level S(t) after the Gaussian function fitting, to the peak detection portion 422.

The peak detection portion 422 detects the peak (the maximum point) and the time of the total activity level S(t) after the Gaussian function fitting. For example, in the example of FIG. 8, the peak detection portion 422 detects a peak value a1 and a peak value a2. The peak value a1 and the peak value a2 each correspond to the “feature point” of the present disclosure.

In addition, the peak detection portion 422 detects a peak time tp1 of the peak value a1, and a peak time tp2 of the peak value a2. The peak detection portion 422 outputs the peak time tp1 and peak time tp2 of the total activity level S(t), to the start-end time fixing portion 423.

The start-end time fixing portion 423 uses the peak time tp1 and the peak time tp2 and fixes a start time and an end time that determine the operation estimating time range. More specifically, the start-end time fixing portion 423 sets a range setting time d1 with respect to the peak time tp1. The range setting time d1 is set based on the spread (e.g., the distribution or the like) of the waveform of the total activity level S (t) at a position at which the peak value a1 is generated, for example. The start-end time fixing portion 423, by subtracting the range setting time d1 from the peak time tp1, sets an estimation range start time t1s with respect to the peak value a1. The start-end time fixing portion 423, by adding the range setting time d1 to the peak time tp1, sets an estimation range end time t1e with respect to the peak value a1. Then, the start-end time fixing portion 423 sets time from the estimation range start time t1s to the estimation range end time t1e, as an operation estimating time range PD1.

Similarly, the start-end time fixing portion 423 sets a range setting time d2 with respect to the peak time tp2. The range setting time d2 is set based on the spread (e.g., the distribution or the like) of the waveform of the total activity level S (t) at a position at which the peak value a2 is generated, for example. The start-end time fixing portion 423, by subtracting the range setting time d2 from the peak time tp2, sets an estimation range start time t2s with respect to the peak value a2. The start-end time fixing portion 423, by adding the range setting time d2 to the peak time tp2, sets an estimation range end time t2e with respect to the peak value a2. Then, the start-end time fixing portion 423 sets time from the estimation range start time t2s to the estimation range end time t2e, as an operation estimating time range PD2.

It is noted that, when a plurality of feature points by a plurality of actions are used for identification, fitting is performed with a function configured by the sum of a plurality of Gaussian functions, which thus determines a range in which the measurement signal yCH(t) is extracted. As an example, in a case in which two feature points by two actions shown in FIG. 8 are used to identify one action, fitting is performed with a function configured by the sum of the Gaussian functions of two waveforms, which thus determines the range setting times d1 and d2.

The start-end time fixing portion 423 outputs the operation estimating time range PD1 and the operation estimating time range PD2, to the arithmetic portion 43.

The identification device 4311 and the identification device 4312 identify an operation, by using the measurement signals yCH1(t) to yCH16(t) in the operation estimating time range PD1 and the operation estimating time range PD2.

The identification device 4311 and the identification device 4312 identify the operation on different conditions. In other words, the identification device 4311 and the identification device 4312 execute identification to be used for operation estimation in different categories. The conditions of the identification, and the identification criteria for the conditions of the identification, are stored in the storage portion 50 and include information learned in advance. It is to be noted that, even during learning in advance, the same or similar method as during the identification described above is used for a setting of the time range of learning data.

For example, the identification device 4311 can be configured to identify five fingers. Specifically, the normative signal (e.g., learning information) of the measurement signals yCH1(t) to yCH16(t) according to the motion of the five fingers obtained by the learning is stored in the storage portion 50. The identification device 4311 is configured to compare the measurement signals yCH1(t) to yCH16(t) with the normative signal, and to identify a finger that has most likely been moved, from a comparison result.

In contrast, the identification device 4312 can be configured to identify raising and lowering of a finger. Specifically, the normative signal (learning information) of the measurement signals yCH1(t) to yCH16(t) according to the motion of the raising and lowering of a finger obtained by the learning is stored in the storage portion 50. The identification device 4312 compares the measurement signals yCH1(t) to yCH16(t) with the normative signal, and identifies a motion that has most likely been moved, from a comparison result.

The identification device 4311 and the identification device 4312 can both be configured to output an identification result to the determination portion 432.

The determination portion 432 then determines an operation by using the identification result of the identification device 4311 and the identification result of the identification device 4312. For example, the determination portion 432 determines which finger moved in which direction by using the identification result of the five fingers of the identification device 4311 and the identification result of an up-and-down motion of the identification device 4312.

In this manner, the operation apparatus 10 is configured to estimate an operation with a finger, by using the configuration and the processing for the present embodiment. At this time, as described above, the estimation is executed by use of a portion (e.g., the operation estimating time range PD1 and the operation estimating time range PD2) including the feature point showing that an operation by the measurement signals yCH1(t) to yCH16(t), that is, the sensor signals, has been performed, and obtaining the amplitude according to the operation. As a result, the operation apparatus 10 uses a measurement signal (a sensor signal) in a range that has a significant effect on improving the accuracy of the estimation, and does not use a measurement signal (a sensor signal) in a range that has little effect on improving the accuracy of the estimation or can be an error factor. Therefore, the operation apparatus 10 is able to estimate an operation with a finger with high accuracy.

In addition, in this configuration and processing, the operation apparatus 10 identifies an operation for each category, by using a plurality of identification devices, and subsequently estimates the operation integrally. Accordingly, the operation apparatus 10 reduces a load of each identification device with respect to identification, and more reliably and rapidly performs identification. Therefore, the operation apparatus 10 more reliably and rapidly estimates an operation.

In addition, in this configuration and processing, a plurality of sensors are worn on both the front surface 911 and back surface 912 of a wrist. As a result, the movement (e.g., the displacement of the surface of a skin) of the tendon of the wrist by the operation with a finger can be detected with higher accuracy, compared with a case in which a plurality of sensors are worn on only the front surface 911 of the wrist or only the back surface 912 of the wrist. Therefore, the operation apparatus 10 is configured to estimate an operation with a finger with higher accuracy.

(2) In a case of performing operation estimation (identification, determination) without using operation estimation time by the Gaussian function fitting

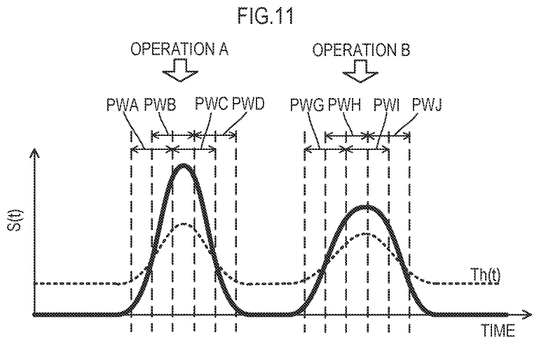

FIG. 11 is a graph illustrating a concept of estimation. In FIG. 11, respective sections set by a horizontal axis indicating time, a vertical axis indicating a value of the total activity level S(t), a solid line indicating time characteristics of the total activity level S(t), a dotted line indicating time characteristics of a threshold value Th(t), and a dashed line correspond to a plurality of time windows PWA, PWB, PWC, PED, PWG, PWH, PWI, and PWJ.

The arithmetic portion 43 is configured to set the plurality of time windows for estimation (e.g., for identification). The plurality of time windows are set at a predetermined time length. The time length of a time window is longer than a sampling period in which identification is performed over time. In other words, the time length of a time window is set so that a plurality of times of identification may be performed during time of one time window.

In addition, the plurality of time windows are set in a predetermined arrangement on a time axis. For example, in the exemplary aspect of FIG. 11, time windows adjacent on the time axis partially overlap with each other. Specifically, the time window PWA and the time window PWB are set so that a second half time of the time window PWA and a first half time of the time window PWB may overlap with each other. The time window PWC and subsequent windows are set similarly. For example, in a case in which the time length of the plurality of time windows is 50 msec., adjacent time windows are set by shifting the time by 25 mse.

It is noted that the time length and arrangement (e.g., the degree of overlap) of the plurality of time windows are not limited to this example, and the adjacent time windows do not need to overlap with each other in alternative aspects.

The identification device 4311 and the identification device 4312 are configured to compare the total activity level S(t) and the threshold value Th(t) for action detection at each timing of identification. The identification device 4311 and the identification device 4312, when the total activity level S(t) is equal to or greater than the threshold value Th(t), set a flag indicating the presence of an action. The identification device 4311 and the identification device 4312, when the total activity level S(t) is less than the threshold value Th(t), set a flag indicating the absence of an action.

In addition, the identification device 4311 and the identification device 4312, at the timing of setting the flag indicating the presence of an action, perform comparison with the normative signal described above and identify the operation.

The identification device 4311 and the identification device 4312 output the flag of the presence or absence of an action, and an identified operation, to the determination portion 432.

The determination portion 432 individually determines the identified operation with respect to each output of the identification device 4311 and the identification device 4312. Hereinafter, although a case of the identification device 4311 is shown as an example, the same applies to a case of the identification device 4312.

The determination portion 432 then divides the flag of the presence or absence of an action and the identification result of the operation that are sequentially obtained from the identification device 4311 into each of the plurality of time windows. The determination portion 432 classifies the flag of the presence or absence of an action and the identification result of the operation for each of the plurality of time windows.

The determination portion 432, at all identification timing points in the time windows, when the flag indicating the presence of an action matches the identification result of the operation, determines the identification result of the operation with respect to this time window.

For example, in the exemplary aspect of FIG. 11, in the time window PWB and the time window PWC, the flag indicates the presence of an action at all the identification timing points. At this time, when all identification results in the time window PWB result in the operation A, the estimation result of an operation with respect to the time window PWB falls into the operation A. Similarly, when all identification results in the time window PWC result in the operation A, the estimation result of an operation with respect to the time window PWC falls into the operation A.

In addition, in the case of FIG. 11, in the time windows PWH and PWI, the flag indicates the presence of an action at all the identification timing points. At this time, when all identification results in the time window PWH result in the operation B, the estimation result of an operation with respect to the time window PWH falls into the operation B. Similarly, when all identification results in the time window PWI result in the operation B, the estimation result of an operation with respect to the time window PWI falls into the operation B.

In contrast, the determination portion 432, when the flag indicating the presence of an action and the flag indicating the absence of an action are mixed in one time window, even with the flag indicating the presence of an action, discards the identification result of the operation with respect to this time window. In other words, the determination portion 432 determines no identification result to this time window.

For example, in the exemplary aspect of FIG. 11, in the time window PWJ, the flag indicating the presence of an action and the flag indicating the absence of an action are mixed. At this time, even when the identification result of the timing of the flag indicating the presence of an action in the time window PWJ results in the operation B, no identification result is determined with respect to the time window PWJ.

In addition, the determination portion 432, at all the identification timing points in the time windows, even with the flag indicating the presence of an action, when the flag does not match the identification result of an operation, discards these identification results. In other words, the determination portion 432 determines no identification result to this time window.

For example, in the exemplary aspect of FIG. 11, in the time window PWI, the flag indicates the presence of an action at all the identification timing points. At this time, when the identification result in the time window PWI is mixed between the operation B and the others, no identification result is determined with respect to the time window PHI.

In addition, the determination portion 432, when the flag indicates the absence of an action at all the identification timing points in the time windows, determines no identification result to this time window.

For example, in the exemplary aspect of FIG. 11, in the time windows PWA, PWD, and PWG, the flag indicates the absence of an action at all the identification timing points. Therefore, no identification result is determined with respect to the time windows PWA, PHD, and PWG.

By performing such processing, the arithmetic portion 43 can estimate an operation. Then, the arithmetic portion 43, even when performing no setting of operation estimation time by the Gaussian function fitting, can further be configured to estimate an operation. As a result, the arithmetic portion 43 more rapidly estimates an operation.

At this time, the normative signal and the threshold value Th(t) for estimation, as described above, are set by use of the learning estimating time range that has been set by the Gaussian function fitting. Therefore, a comparable used for estimation is highly accurate, and the arithmetic portion 43 achieves highly accurate estimation.

Furthermore, with use of this method, a combined operation can be estimated. The combined operation is configured by combining a plurality of operations, and is identified as a specific operation. For example, (lowering of a finger)+(raising of the same finger as the lowered finger)=(a click operation). At this time, time from (lowering of a finger) to (raising of the finger) is also a determining factor to identify a specific operation.

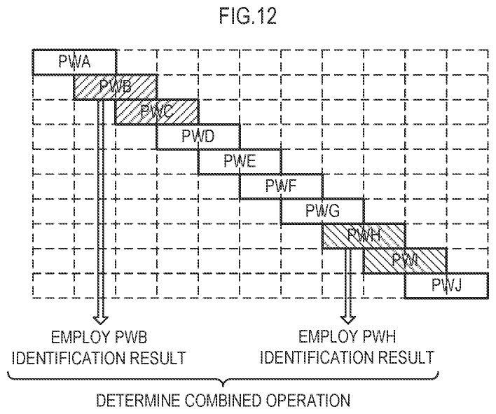

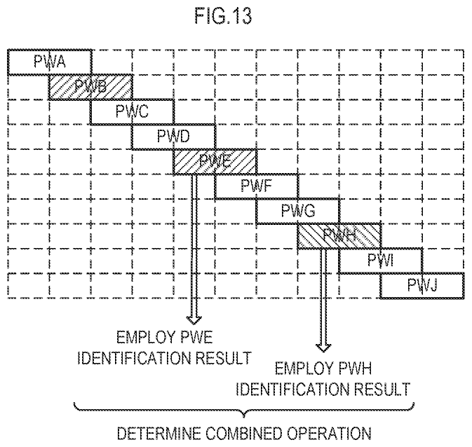

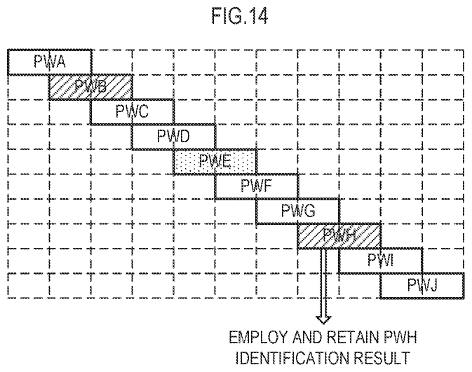

FIG. 12, FIG. 13, and FIG. 14 show a concept of exemplary aspects in which a combined operation is determined. In FIG. 12, FIG. 13, and FIG. 14, each frame represents an own time window. In addition, a hatched time window indicates that the identification result of an operation as a time window is obtained, and operation content varies, depending on the type of hatching.

In the exemplary aspect of FIG. 12, the same operation (the operation A, for example) is identified in the time window PWB and the time window PWC. In such a case, the determination portion 432 employs the identification result of the time window PWB that first identifies this operation (the operation A, for example). Then, the determination portion 432 discards the identification result of the time window PWC following the time window PWB.

Moreover, the same operation (the operation B, for example) is identified in the time window PWH and the time window PWI. In such a case, the determination portion 432 employs the identification result of the time window PWH that first identifies this operation (the operation B, for example). Then, the determination portion 432 discards the identification result of the time window PWI following the time window PWH.

Then, the determination portion 432 determines a specific operation by combining the identification result (the operation A) of the time window PWB and the identification result (the operation B) of the time window PWH. For example, when the operation A is (lowering of a right index finger), and the operation B is (raising of the right index finger), the determination portion 432 determines (a click operation by the right index finger) from these identification results.

At this time, the determination portion 432 counts time starting from the time window PWB, and, when obtaining no identification result of the next operation within a determination retention time period in accordance with identification of the specific operation, determines the operation identified in the time window PWB, as a single operation. In other words, the determination portion 432, when obtaining no identification result of the next operation within time in accordance with the identification of the specific operation with respect to a time window being a starting point of the specific operation, discards the identification result of the operation identified in the time window set as the starting point. It is to be noted that, when no identification result of the next operation within time in accordance with the identification of the specific operation is obtained, the operation identified in the time window set as the starting point is also able to be detected as a single operation.

It is noted that the determination criteria for such a specific operation are able to be learned in the same way as the learning of the individual operation described above, and are stored in the storage portion 50. The determination portion 432, referring to this storage content, determines the specific operation.

In the exemplary aspect of FIG. 13, the same operation (the operation A, for example) is identified in the time window PWB and the time window PWE. In such a case, the determination portion 432 employs the identification result of the time window PWE that finally identifies this operation (the operation A, for example). Then, the determination portion 432 discards the identification result of the time window PWB. In other words, the determination portion 432, in a case in which the same operation is identified in a plurality of time windows that are not adjacent to each other on a time axis, employs the identification result of the time window that finally identifies the operation.

In addition, in the exemplary aspect of FIG. 13, in the time window PWH, a different operation (the operation B, for example) from the time window PWE is identified. Since no identification result of the same operation as the time window PWH within a predetermined time period before and after the time window PWH is present, the determination portion 432 employs the identification result of the time window PWH.

Then, the determination portion 432 determines the specific operation by combining the identification result (the operation A) of the time window PWE and the identification result (the operation B) of the time window PWH.

Moreover, in the exemplary aspect of FIG. 14, the same operation (the operation A, for example) is identified in the time window PWB and the time window PWH. Then, in the time window PWE, since the flag indicating the absence of an action is partially included, even when a different operation (the operation B, for example) from the time window PWB and the time window PWH is performed, the identification result is not obtained.

In such a case, the determination portion 432 determines a combined operation, by the identification result of the time window PWB and the identification result of the time window PWH. The time window PWB and the time window PWH have the same identification results, and are spaced apart on the time axis. Therefore, the determination portion 432 employs the identification result (the operation A) of the time window PWH, and discards the identification result of the time window PWB. Then, the determination portion 432 stores the identification result of the time window PWH for the determination retention time period, and retains the determination of the specific operation.

By performing such processing, the arithmetic portion 43 can identify (e.g., estimate) a combined operation. At this time, even when no setting of operation estimation time by the Gaussian function fitting is performed, an operation is able to be estimated. As a result, the arithmetic portion 43 is able to more rapidly estimate an operation. In addition, as described above, a comparable used for estimation is highly accurate, and the arithmetic portion 43 is able to achieve highly accurate estimation.

It is noted that the above exemplary embodiment shows a case in which the number of sensors is 16. However, as also described above, the number of sensors is not limited to this and may be two or more. For example, the number of sensors may be set to a predetermined number, based on the number of fingers to detect an operation, the type of motion of the finger to be estimated, and the like.

In addition, the above exemplary embodiment shows an aspect in which a chart is used as the total activity level S(t). However, with use of a total value of the amplitude of the measurement signals yCH1(t) to yCH16(t), the total activity level S (t) is calculable.

Moreover, the above exemplary embodiment shows an aspect in which two identification devices are used. However, the number of identification devices is not limited to this and may be appropriately set according to identification conditions. For example, when horizontal movement is identified as a motion of a finger, in addition to vertical movement, the operation apparatus may further add an identification device that identifies the horizontal movement. It is to be noted that it is also possible to use one identification device to identify all.

(Operation Estimation Method)

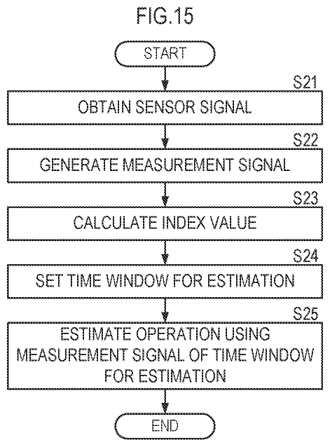

FIG. 15 is a flow chart showing an example of the operation estimation method according to the first exemplary embodiment. It is to be noted that the processing shown in FIG. 15 shows a case in which the above time window is used. The estimation method using the operation estimating time range using the Gaussian function fitting is able to be achieved by replacing the term of learning in the learning method shown in the above FIG. 10, with estimation.

The operation apparatus 10 generates a sensor signal according to the movement (the displacement of the surface of a skin) of the tendon of a wrist by the operation with a finger, by the plurality of sensors 201 to 216 (S21). The operation apparatus 10, by using sensor signals of the plurality of sensors, generates the measurement signals yCH1(t) to yCH16(t), respectively (S22).

The operation apparatus 10, by using the measurement signals of the plurality of sensors, calculates the total activity level S(t) being a range setting index (an index value) (S23). The operation apparatus 10 sets a time window for estimation (S24). The operation apparatus 10, by using the measurement signals yCH1(t) to yCH16(t) in the time window for estimation, estimates the operation (S25).

In operation estimation (S25), as described above, it is also possible to estimate a combined operation from a plurality of identification results at a plurality of time points and further from a temporal connection between the plurality of identification results. In other words, in a case in which the plurality of identification results satisfy a condition that shows one (one type of) operation, this one operation is estimated by use of the plurality of these identification results. For example, in a case of identification of the lowering of a certain finger subsequently followed by identification of the raising of the same finger, a tap operation is estimated.

On the other hand, in a case in which the plurality of identification results do not satisfy the condition that shows one (e.g., one type of) operation, each of the plurality of identification results is used as an individual identification result to estimate each individual operation. For example, in a case of identification of the lowering of a certain finger subsequently followed by identification of the raising of a different finger, these operations are estimated as individual operations.

(Example of Application Target of Operation Estimation)

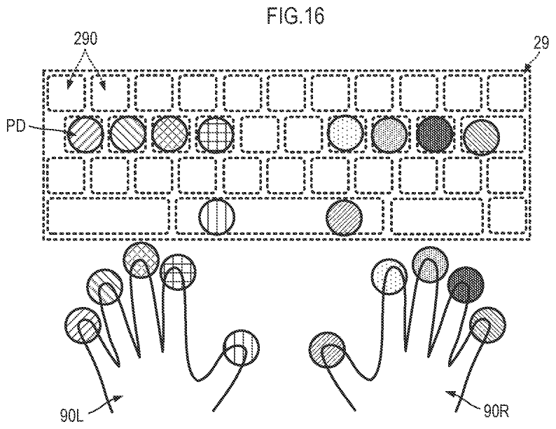

FIG. 16 is a view showing an example of an application target of the operation apparatus according to the present exemplary embodiment. In FIG. 16, each hatched circle indicates a default position PD of each finger. As shown in FIG. 16, the operation with a finger that is estimated by the operation apparatus 10 is able to be used for an input to a virtual keyboard 29, for example.

Specifically, the virtual keyboard 29 is arranged with a plurality of virtual keys 290. Coordinates are set to the plurality of virtual keys 290, respectively. The default position PD of each finger is set to the virtual keyboard 29. The default position is set to each finger, that is, each of the five fingers of the right hand 90R and each of the five fingers of the left hand 90L. Such default positions PD are set mainly by prior learning, for example. A moved finger and the motion are estimated by the operation apparatus 10. This motion is assigned to movement of a finger that operates the virtual keyboard 29, a key-pressing action, or the like. As a result, in the virtual keyboard 29, it is possible to estimate and detect which virtual key 290 has been pressed.

Accordingly, even without a physical character keyboard, the operation apparatus 10, by detecting a motion of a finger or an operation in the air, on a desk, or the like, is able to input text to an electronic device (a smartphone, a PC, or the like, for example) paired to the operation apparatus 10. In other words, the operation apparatus 10 functions as an input device.

### Second Exemplary Embodiment

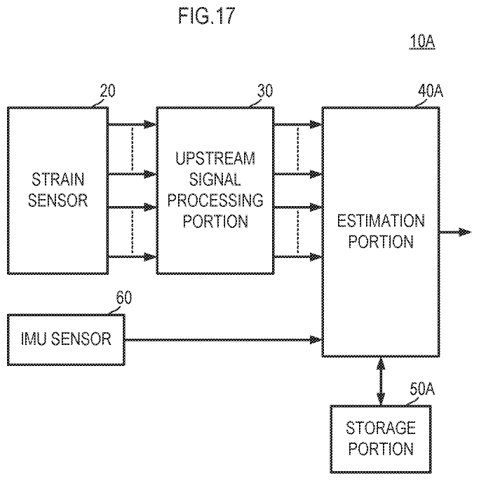

An operation estimation technology according to a second exemplary embodiment will be described with reference to the drawings. FIG. 17 is a functional block diagram showing an example of a configuration of an operation apparatus according to the second exemplary embodiment.

As shown in FIG. 17, an operation apparatus 10A according to the second exemplary embodiment is different in addition of an IMU sensor 60 and processing of an estimation portion 40A from the operation apparatus 10 according to the first exemplary embodiment. It is noted that other configurations of the operation apparatus 10A are the same as or similar to the configurations of the operation apparatus 10, and a description of the same or similar configurations will be omitted.

As further described herein, the operation apparatus 10A includes an estimation portion 40A, a storage portion 50A, and an IMU sensor 60. In an exemplary aspect, the IMU sensor 60 includes a triaxial acceleration sensor and a triaxial angular velocity sensor. Moreover, the IMU sensor 60 is configured to be worn on a wrist and measures a motion of the wrist. The IMU sensor 60 outputs an IMU measurement signal to the estimation portion 40A.

The estimation portion 40A estimates an operation with a finger, by using the IMU measurement signal together with the measurement signals yCH1(t) to yCH16(t) of the plurality of sensors 201 to 216. At this time, the storage portion 50A, for the IMU measurement signal, stores a normative signal for IMU measurement signals, and the determination criteria for operation estimation. The estimation portion 40A estimates the operation with a finger, by referring to the normative signal and the determination criteria for operation estimation that are stored in the storage portion 50A and using the IMU measurement signal.

At this time, the estimation portion 40A, for example, is also configured to use a separate identification device for IMU measurement signals from the identification devices for the measurement signals yCH1(t) to yCH16(t) of the plurality of sensors 201 to 216. With use of such separate identification devices, it is possible to reduce a load on each identification device and improve the accuracy of operational estimation.

### Third Exemplary Embodiment

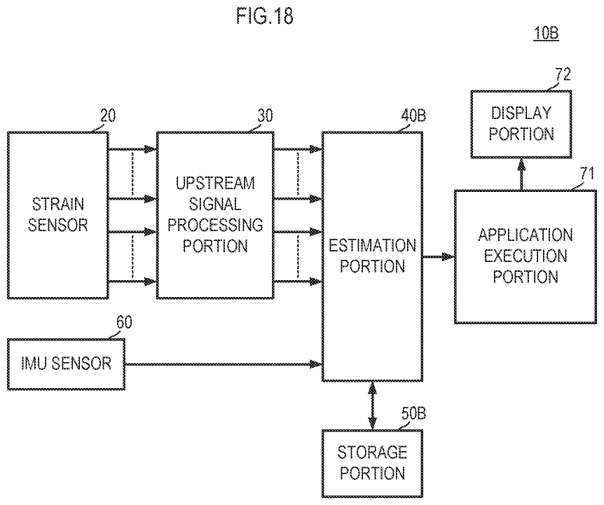

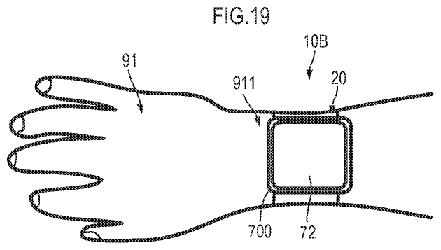

An operation estimation technology according to a third exemplary embodiment will be described with reference to the drawings. FIG. 18 is a functional block diagram showing an example of a configuration of an operation apparatus according to the third exemplary embodiment. FIG. 19 is a view showing a wearing example of the operation apparatus according to the third exemplary embodiment.

As shown in FIG. 18, an operation apparatus 10B according to the third exemplary embodiment is different from the operation apparatus 10A according to the second exemplary embodiment in that an application execution portion 71 and a display portion 72 are provided. It is noted that other configurations of the operation apparatus 10B are the same as or similar to the configurations of the operation apparatus 10, and a description of the same or similar configurations will be omitted. It is also noted that an estimation portion 40B and a storage portion 50B of the operation apparatus 10B are the same as the estimation portion 40A and storage portion 50A of the operation apparatus 10A, and a description will be omitted.

The operation apparatus 10B includes an application execution portion 71 and a display portion 72. The application execution portion 71 is configured by a CPU, a memory that stores an application to be executed by the CPU, and the like, for example. An operation estimation result is inputted into the application execution portion 71.

The application execution portion 71 executes, for example, a document creation application, an email application, an SNS application, or the like. At this time, the application execution portion 71 estimates character input from a key operation state detected by the operation estimation result and reflects the result in various applications. The application execution portion 71 outputs an execution result of an application to the display portion 72. The display portion 72 displays the execution result of an application.

In such a manner, for example, as shown in FIG. 19, the operation apparatus 10B includes a structure similar to a smartwatch. In other words, as shown in FIG. 19, the operation apparatus 10B includes a housing 700. The housing 700 has a size large enough to be worn on a wrist. The housing 700 is mounted on a top of the strain sensor 20, and is connected to the sensor 20.

The display portion 72 is disposed on a front surface of the housing 700. The housing 700 houses function portions other than the strain sensor 20 and the display portion 72 in the operation apparatus 10B.

### Fourth Exemplary Embodiment

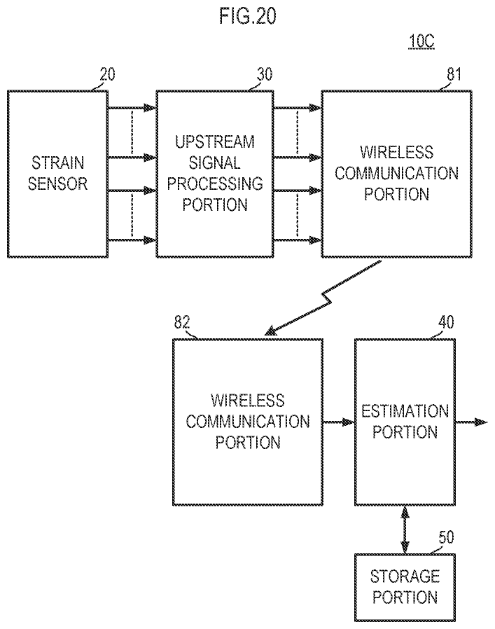

An operation estimation technology according to a fourth exemplary embodiment will be described with reference to the drawings. FIG. 20 is a functional block diagram showing an example of a configuration of an operation apparatus according to the fourth exemplary embodiment.

As shown in FIG. 20, an operation apparatus 10C according to the fourth exemplary embodiment is different from the operation apparatus 10 according to the first exemplary embodiment in that a wireless communication portion 81 and a wireless communication portion 82 are provided. It is noted that other configurations of the operation apparatus 10C are the same as or similar to the configurations of the operation apparatus 10, and a description of the same or similar configurations will be omitted.

According to the exemplary aspect, the operation apparatus 10C includes a wireless communication portion 81 and a wireless communication portion 82. The wireless communication portion 81 is connected to an output side of the upstream signal processing portion 30. The wireless communication portion 82 is connected to an input side of the estimation portion 40.

Moreover, the wireless communication portion 81 is configured to send the measurement signals yCH1(t) to yCH16(t) of the plurality of sensors 201 to 216 to the wireless communication portion 82. The wireless communication portion 82 outputs the received measurement signal yCH1(t) to yCH16(t) to the estimation portion 40.

With such a configuration, the operation apparatus 10C is able to separate a configuration up to generating the measurement signals yCH1(t) to yCH16(t) and a configuration to estimate an operation. As a result, a part worn on a wrist is able to be reduced, and the operation apparatus 10C is able to further significantly reduce a sense of discomfort of a wearer and further improve operability.

It is noted that the configuration to be separated by wireless, although being not limited to being in a location shown in the present exemplary embodiment, sends and receives the measurement signals yCH1(t) to yCH16(t) being digital signals with relatively clear waveforms, for example, in the configuration of the present exemplary embodiment. Therefore, occurrence of incorrect estimation due to noise is able to be more reduced than transmitting and receiving a sensor signal.

### Fifth Exemplary Embodiment

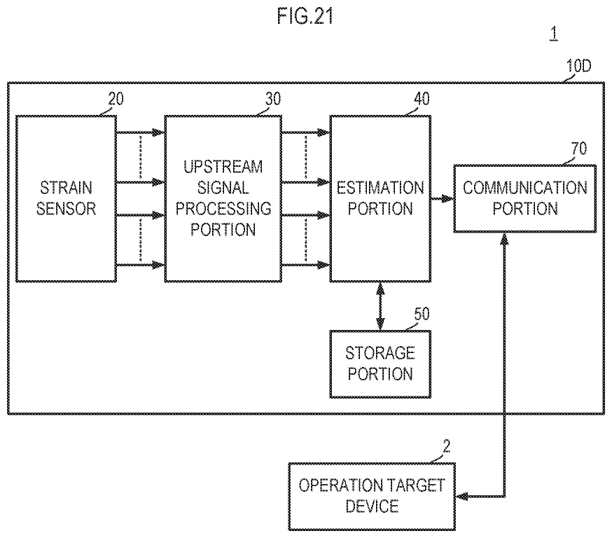

An operation estimation technology according to a fifth exemplary embodiment will be described with reference to the drawings. FIG. 21 is a functional block diagram showing an example of a configuration of an operation apparatus according to the fifth exemplary embodiment.

As shown in FIG. 21, an operation estimation system 1 includes an operation apparatus 10D and an operation target device 2. The operation apparatus 10D is different from the operation apparatus 10 according to the first exemplary embodiment in that a communication portion 70 is provided. It is noted that other configurations of the operation apparatus 10D are the same as or similar to the configurations of the operation apparatus 10, and a description of the same or similar configurations will be omitted.

The communication portion 70 is connected to an output side of the estimation portion 40, and receives an input of an estimation result of an operation from the estimation portion 40. The communication portion 70 has a wireless communication function, for example, and is configured to communicate with the operation target device 2. The communication portion 70 sends the estimation result of an operation, to the operation target device 2.

The operation target device 2, by using the estimation result of an operation, executes a predetermined application (e.g., an application executed by the application execution portion 71 shown in the above embodiment, or the like, for example).

In this manner, the above estimation of the operation with a finger is not limited to the use by an apparatus alone and is also able to be used as a system.

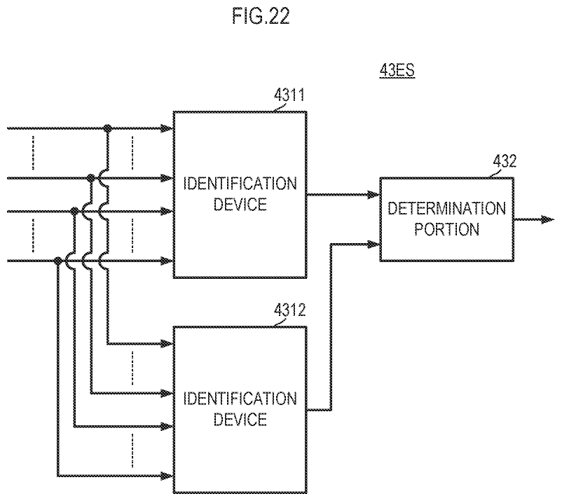

It is noted that the above description shows an aspect in which the operation apparatus includes both a “learning” function and an “estimation” function. However, the operation apparatus may include only the “estimation” function. FIG. 22 is a functional block diagram showing an example of a configuration of an arithmetic portion that only estimates an operation.

As shown in FIG. 22, an arithmetic portion 43ES of the operation apparatus that has no learning function and only performs estimation includes an identification device 4311, an identification device 4312, and a determination portion 432. In other words, the arithmetic portion 43ES includes no learning portion 433 in the arithmetic portion 43.

In such a case, learning is performed by another operation apparatus including at least the learning portion 433 in the same configuration as this operation apparatus. Then, the operation apparatus that has only the estimation function stores a learning result in the storage portion 50 in advance, and performs estimation of an operation by using a stored learning result.

In addition, the operation apparatus that has only the estimation function, when having a communication function with the outside, is configured to appropriately obtain the learning result stored in an external server or the like, and estimate the operation.

Each of the above exemplary embodiments describes an operation input such as a key with a finger, as the main focus. However, the configuration and processing of each of the exemplary embodiments are not limited to a key input. For example, the exemplary aspects of present invention are also applicable to an apparatus in other fields, such as a game machine that is operated by moving fingers.

In addition, the configuration and processing of each of the above exemplary embodiments can be appropriately combined as would be appreciated to one skilled in the art, and advantageous functions and effects according to each combination can be obtained.

### REFERENCE SIGNS LIST

1: operation estimation system
2: operation target device
10, 10A, 10B, 10C, 10D: operation apparatus
20: strain sensor
29: virtual keyboard
30: upstream signal processing portion
40: estimation portion
40A: estimation portion
40B: estimation portion
41: index value calculation portion
42: range setting portion
43, 43ES: arithmetic portion
50, 50A, 50B: storage portion
60: IMU sensor
70: communication portion
71: application execution portion
72: display portion
81, 82: wireless communication portion
90L: left hand
90R: right hand
91: back of hand
201 to 216: sensor
290: virtual key
411: chart generation portion
412: total activity level calculation portion
421: Gaussian function fitting portion
422: peak detection portion
423: start-end time fixing portion
432: determination portion
700: housing
911: front surface
912: back surface
4311, 4312: identification device

What is claimed:

1. An operation apparatus comprising:
a plurality of sensors configured to be worn on a wrist and to output a sensor signal based on a displacement of a body surface of the wrist;
a range setting portion configured to set an operation learning time range that includes a time of a feature point of the sensor signal of the plurality of sensors; and
an arithmetic portion configured to learn an operation based on the sensor signal of the plurality of sensors in the operation learning time range.

2. The operation apparatus according to claim 1, further comprising:
an index value calculator configured to calculate a range setting index, by using a magnitude of the sensor signal of the plurality of sensors,
wherein the range setting portion is further configured to set the operation learning time range, with a feature point of the range setting index, as the feature point of the sensor signal.

3. The operation apparatus according to claim 2, wherein the index value calculator is further configured to calculate the range setting index as a total value of the magnitude of the sensor signal of the plurality of sensors.

4. The operation apparatus according to claim 2, wherein the range setting portion is further configured to detect the feature point from time characteristics of the range setting index.

5. The operation apparatus according to claim 4, wherein the range setting portion is further configured to set the feature point as a detected peak value of the range setting index.

6. The operation apparatus according to claim 2, wherein the range setting portion is further configured to set the operation learning time range as a predetermined time range that includes the time of the feature point.

7. The operation apparatus according to claim 6, wherein the range setting portion is further configured to set the predetermined time range including the time of the feature point, by spread of the time characteristics of the range setting index.

8. The operation apparatus according to claim 2, wherein the range setting portion is configured to:
perform a fitting based on a normal distribution on the time characteristics of the range setting index,
detect the feature point, and
set the operation learning time range.

9. An operation apparatus comprising:
a plurality of sensors configured to be worn on a wrist and to output a sensor signal based on a displacement of a body surface of the wrist;
a range setting portion configured to set an operation estimating time range that includes time of a feature point of the sensor signal of the plurality of sensors; and
an arithmetic portion configured to estimate an operation based on the sensor signal of the plurality of sensors in the operation estimating time range.

10. An operation apparatus comprising:
a plurality of sensors configured to be worn on a wrist and to output a sensor signal based on a displacement of a body surface of the wrist;
a total activity level calculator configured to calculate a total activity level obtained from a total of intensity of the sensor signal of the plurality of sensors;
a range setting portion configured to set a time window for operation estimation; and
an arithmetic portion configured to estimate an operation based on the total activity level in the time window and the sensor signal.

11. The operation apparatus according to claim 10, wherein the arithmetic portion is further configured to estimate the operation based on a magnitude of the total activity level in a plurality of time periods in the time window and an identification result of the operation by the sensor signal.

12. The operation apparatus according to claim 11, wherein the arithmetic portion is further configured to determine the identification result as the operation in the time window when the total activity level for all the time periods in the time window is equal to or greater than a threshold value for action detection and all the time periods have a same identification result.

13. The operation apparatus according to claim 11, wherein the arithmetic portion is further configured to retain the identification result in a first time window in the plurality of consecutive time windows and to discard the identification result in other time windows when the same identification result of the operation is determined in a plurality of consecutive time windows.

14. The operation apparatus according to claim 13, wherein the arithmetic portion, in a retention time period of the identification result in the plurality of time windows, is further configured to keep the identification result in a last time window and to discard the identification result in other time windows when the same identification result of the operation is determined in a plurality of inconsecutive time windows.

15. The operation apparatus according to claim 10, wherein the arithmetic portion includes:
a plurality of identification devices that are each configured to identify an operation on different conditions to each sensor signal of the plurality of sensors; and
a determination portion configured to determine the operation based on a result identified by the plurality of identification devices.

16. The operation apparatus according to claim 15, wherein the plurality of identification devices are configured identify the operation based on a relationship between previously learned operation content and the sensor signal of the plurality of sensors.

17. The operation apparatus according to claim 10, wherein the plurality of sensors include:
a front side sensor group configured to be worn on a front side of the wrist; and
a back side sensor group configured to be worn on a back side of the wrist.

18. The operation apparatus according to claim 10, wherein the plurality of sensors are configured to output the sensor signal based on the displacement of the body surface of the wrist that occurs by a motion of at least one of a hand and a finger.

19. The operation apparatus according to claim 10, wherein the plurality of sensors are piezoelectric sensors having an electrode disposed on a piezoelectric film with flexibility.

20. The operation apparatus according to claim 10, further comprising a display configured to display an estimation result of the operation.

21. The operation apparatus according to claim 10, further comprising an application execution portion configured to execute an application based on the estimation result of the operation.

22. The operation apparatus according to claim 10, further comprising a communication portion configured to send the estimation result of the operation to an external operation target device.
